(19) United States
(12) Patent Application Publication
(10) Pub. No.: US 2019/0112150 A1
(43) Pub. Date: Apr. 18, 2019

(54) ELEVATOR SYSTEM

(71) Applicant: Mitsubishi Electric Corporation, Chiyoda-ku (JP)

(72) Inventor: Tatsuya UETSU, Aichi (JP)

(73) Assignee: Mitsubishi Electric Corporation, Chiyoda-ku (JP)

(21) Appl. No.: 16/090,313

(22) PCT Filed: Jun. 20, 2016
(86) PCT No.: PCT/JP2016/068261

§ 371 Date: Oct. 1, 2018

(51) Int. Cl. B66B 5/00; E05B 49/00; G16H 70/60

(52) U.S. Cl. CPC B66B 5/0012; B66B 1/2408; G16H 70/60; E05B 49/00

(57) ABSTRACT

A dynamic state management system capable of operating appropriately according to a health condition of a user. The dynamic state management system includes: a reading device provided around an entrance of a management area, the reading device reading identification information from an identifier of a user; and a determination device configured to determine whether or not to permit passage through the entrance based on the identification information read by the reading device and health condition information collected from a wearable terminal of a user corresponding to the identification information. By adopting this configuration, a determination is made in accordance with health condition information collected from a wearable terminal of a user. Therefore, the dynamic state management system can operate appropriately according to a health condition of a user.

### TECHNICAL FIELD

The present invention relates to a dynamic state management system and an elevator system.

### BACKGROUND ART

PTL 1 discloses a health condition management system. According to the health condition management system, a health condition of a user can be managed.

### CITATION LIST

### Patent Literature

[PTL 1] Japanese Patent Application Laid-open No. 2003-303239

### SUMMARY OF INVENTION

### Technical Problem

However, the health condition management system described in PTL 1 does not take users in a good health condition into consideration. Therefore, a user in a good health condition may become infected with an infectious disease.

The present invention has been made in order to solve the problem described above. An object of the present invention is to provide a dynamic state management system and an elevator system capable of operating appropriately according to a health condition of a user.

### Solution to Problem

A dynamic state management system according to the present invention includes: a reading device provided around an entrance of a management area, the reading device reading identification information from an identifier of a user; and a determination device configured to determine whether or not to permit passage through the entrance based on the identification information read by the reading device and health condition information collected from a wearable terminal of a user corresponding to the identification information.

An elevator system according to the present invention includes: a plurality of reading devices provided on respective halls of an elevator, the plurality of reading devices reading identification information from an identifier of a user; and a control device configured to control the elevator based on the identification information read by any reading device of the plurality of reading devices and health condition information collected from a wearable terminal of a user corresponding to the identification information.

### Advantageous Effects of Invention

According to these inventions, a determination or control in accordance with health condition information collected from a wearable terminal of a user is performed. As a result, the dynamic state management system and the elevator system can operate appropriately according to a health condition of the user.

### BRIEF DESCRIPTION OF DRAWINGS

FIG. 1 is a diagram for illustrating an example of application of a dynamic state management system according to a first embodiment of the present invention.

FIG. 2 is a diagram for illustrating an outline of control of an electronic lock by the dynamic state management system according to the first embodiment of the present invention.

FIG. 3 is a diagram for illustrating an outline of control of an electronic lock by the dynamic state management system according to the first embodiment of the present invention.

FIG. 4 is a diagram for illustrating health management of a user by the dynamic state management system according to the first embodiment of the present invention.

FIG. 5 is a diagram for illustrating health management of a user by the dynamic state management system according to the first embodiment of the present invention.

FIG. 6 is a flow chart for illustrating an outline of operations of a local controller of the dynamic state management system according to the first embodiment of the present invention.

FIG. 7 is a hardware configuration diagram of the local controller and a center device of the dynamic state management system according to the first embodiment of the present invention.

FIG. 8 is a diagram for illustrating a determination whether or not to permit passage by a dynamic state management system according to a second embodiment of the present invention.

FIG. 9 is a diagram for illustrating a determination whether or not to permit passage by a dynamic state management system according to a third embodiment of the present invention.

FIG. 10 is a diagram for illustrating an elevator system according to a fourth embodiment of the present invention.

FIG. 11 is a diagram for illustrating an outline of control by the elevator system according to the fourth embodiment of the present invention.

FIG. 12 is a diagram for illustrating operations of a car of the elevator system according to the fourth embodiment of the present invention.

FIG. 13 is a diagram for illustrating operations of a control device of the elevator system according to the fourth embodiment of the present invention.

FIG. 14 is a diagram for illustrating operations of the control device of the elevator system according to the fourth embodiment of the present invention.

### DESCRIPTION OF EMBODIMENTS

Embodiments of the present invention will now be described with reference to the accompanying drawings. It should be noted that, in the respective drawings, same or corresponding portions are assigned same reference signs. Redundant descriptions of such portions will be simplified or omitted as appropriate.

### First Embodiment

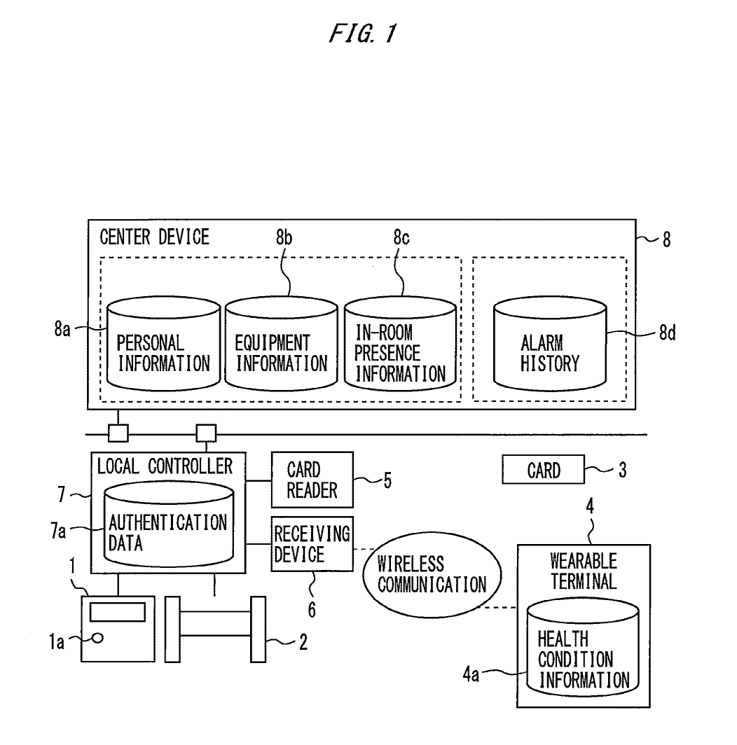

FIG. 1 is a diagram for illustrating an example of application of a dynamic state management system according to a first embodiment of the present invention.

In FIG. 1, a door 1 is provided at an entrance of a management area (not shown). An electronic lock 1a is provided on the door 1. A gate 2 is provided at the entrance of the management area.

A card 3 is carried around by a user. The card 3 stores identification information of the user as an identifier. For example, a wearable terminal 4 is attached to an arm of the user. For example, the wearable terminal 4 is attached to a wrist of the user. The wearable terminal 4 collects a health condition of the user. The wearable terminal 4 is provided with a storage unit 4a. The storage unit 4a stores health condition information of the user.

For example, health condition information is constituted by a “date and time”, a “body temperature 1”, a “body temperature 2”, a “body temperature 3”, a “body temperature 4”, a “body temperature 5”, and a “body temperature 6”.

The “date and time” represents a date and time at which health condition information is collected. A data format of the “date and time” is expressed as “YYYY/MM/DD hh:mm:ss”. “YYYY” represents the year. “MM” represents the month. “DD” represents the day. “hh” represents the hour. “mm” represents the minute. “ss” represents the second.

The “body temperature 1” represents a current body temperature of the user. A data format of the “body temperature 1” is expressed as “NN.N”. When the body temperature of the user is 36.5 degrees, “NN.N” is 36.5. For example, when the body temperature of the user is 37.5 degrees, “NN.N” is 37.5.

The “body temperature 2” represents an average body temperature of the user from a day ago. The “body temperature 2” is an average over 24 hours. For example, when the “date and time” is “2016/01/03 12:00:00”, the “body temperature 2” is an average body temperature of the user over a period from “2016/01/01 12:00:00” to “2016/01/02 11:59:59”. A data format of the “body temperature 2” is the same as that of the “body temperature 1”.

The “body temperature 3” represents an average body temperature of the user from two days ago. The “body temperature 3” is an average over 24 hours. For example, when the “date and time” is “2016/01/03 12:00:00”, the “body temperature 3” is an average body temperature of the user over a period from “2015/12/31 12:00:00” to “2016/01/01 11:59:59”. A data format of the “body temperature 3” is the same as that of the “body temperature 1”.

The “body temperature 4” represents an average body temperature of the user from three days ago. The “body temperature 4” is an average over 24 hours. For example, when the “date and time” is “2016/01/03 12:00:00”, the “body temperature 4” is an average body temperature of the user over a period from “2015/12/30 12:00:00” to “2015/12/31 11:59:59”. A data format of the “body temperature 4” is the same as that of the “body temperature 1”.

The “body temperature 5” represents an average body temperature of the user from four days ago. The “body temperature 5” is an average over 24 hours. For example, when the “date and time” is “2016/01/03 12:00:00”, the “body temperature 5” is an average body temperature of the user over a period from “2015/12/29 12:00:00” to “2015/12/30 11:59:59”. A data format of the “body temperature 5” is the same as that of the “body temperature 1”.

The “body temperature 6” represents an average body temperature of the user from five days ago. The “body temperature 6” is an average over 24 hours. For example, when the “date and time” is “2016/01/03 12:00:00”, the “body temperature 5” is an average body temperature of the user over a period from “2015/12/28 12:00:00” to “2015/12/29 11:59:59”. A data format of the “body temperature 6” is the same as that of the “body temperature 1”.

The dynamic state management system is provided with a card reader 5, a receiving device 6, a local controller 7, and a center device 8.

The card reader 5 is provided around an entrance of a management area as a reading device. The receiving device 6 is provided around the entrance of the management area. The local controller 7 is provided with an authentication data storage device 7a. The center device 8 is provided with a personal information storage device 8a, an equipment information storage device 8b, an in-room presence information storage device 8c, and an alarm history storage device 8d.

For example, in accordance with an issuance of the card 3, the center device 8 causes personal information including identification information to be stored in the personal information storage device 8a by an operation from the outside. In doing so, the identification information is downloaded to the local controller 7 as authentication data. For example, the center device 8 causes equipment information which associates information on the electronic lock 1a, information on the card reader 5, and information on the receiving device 6 with one another to be stored in the equipment information storage device 8b by an operation from the outside. For example, the center device 8 causes equipment information which associates information on the gate 2, information on the card reader 5, and information on the receiving device 6 with one another to be stored in the equipment information storage device 8b by an operation from the outside.

When a user waves the card 3 over the card reader 5, the card reader 5 reads identification information from the card 3. The local controller 7 collates the identification information read by the card reader 5 with authentication data.

When a user approaches the entrance of the management area, the receiving device 6 receives health condition information from the wearable terminal 4 of the user.

When identification information read by the card reader 5 matches any of the pieces of authentication data, the local controller 7 acquires health condition information of a user from the receiving device 6.

The local controller 7 determines, as a determination device, whether or not to permit passage through the entrance of the management area based on health condition information of a user. The local controller 7 controls the electronic lock 1a or the gate 2 based on a result of determination whether or not to permit passage through the entrance of the management area.

The local controller 7 transmits, to the center device 8, passage information which associates the identification information read by the card reader 5, information on the door 1 or the management area, and information on a current date and time with each other. The local controller 7 transmits health condition information of the user acquired from the receiving device 6 to the center device 8.

The center device 8 causes in-room presence information which associates passage information and health condition information with each other to be stored in the in-room presence information storage device 8c. For example, when the door 1 opens after permission for passage from the outside to the inside of the entrance is given, the center device 8 adopts information indicating a “present-in-room state” as information of an in-room presence state. For example, when the door 1 opens after permission for passage from the inside to the outside of the entrance is given, the center device 8 adopts information indicating a “not present-in-room state” as information of the in-room presence state.

The center device 8 issues an alarm based on the information stored in the in-room presence information storage device 8c. The center device 8 causes alarm history which associates alarm information and information on a current date and time with each other to be stored in the alarm history storage device 8d.

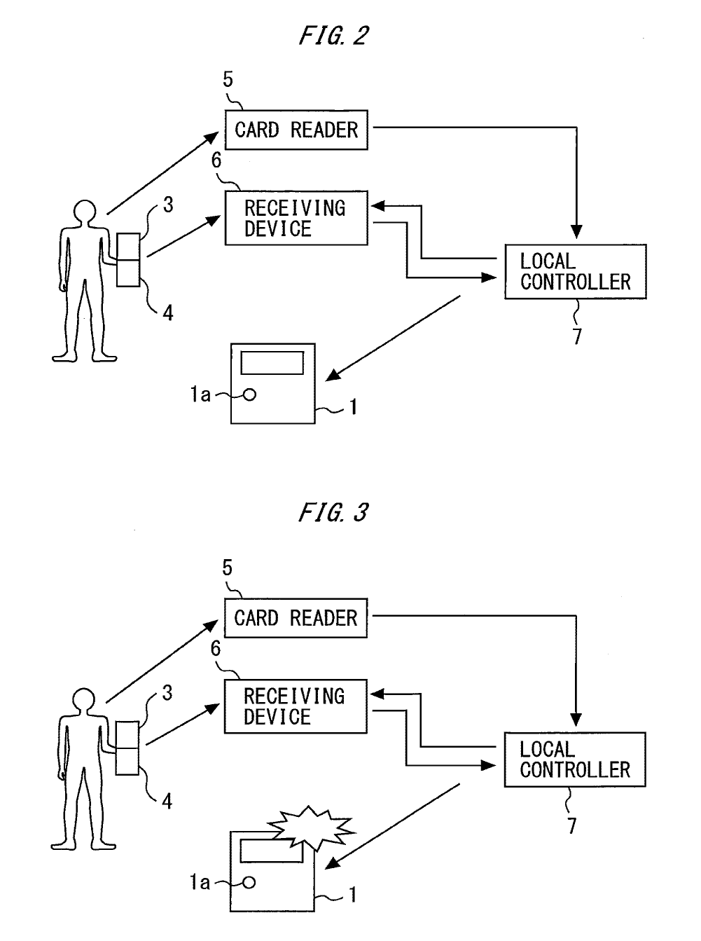

Next, an outline of control of the electronic lock 1a will be described with reference to FIGS. 2 and 3.

FIGS. 2 and 3 are diagrams for illustrating an outline of control of an electronic lock by the dynamic state management system according to the first embodiment of the present invention.

For example, when preventing a disease from spreading inside the management area, a health condition of a user whose body temperature is equal to or higher than 37.5° C. is considered poor. In this configuration, the local controller 7 permits passage from the outside to the inside of the management area when the “body temperature 1” of the user is lower than 37.5. When the “body temperature 1” of the user is equal to or higher than 37.5, the local controller 7 does not permit passage from the outside to the inside of the management area.

For example, when preventing a disease which requires bed rest for a certain period of time from spreading inside the management area, a health condition of a user whose average body temperature was equal to or higher than 37.5° C. on any of the past five days is considered poor. In this configuration, the local controller 7 permits passage from the outside to the inside of the management area when all of the “body temperature 1” to the “body temperature 6” of the user are lower than 37.5. When at least one of the “body temperature 1” to the “body temperature 6” of the user is equal to or higher than 37.5, the local controller 7 does not permit passage from the outside to the inside of the management area.

For example, when preventing a disease from being transmitted inside the management area, a health condition of a user whose body temperature is equal to or higher than 37.0° C. is considered poor. In this configuration, the local controller 7 permits passage from the outside to the inside of the management area when the “body temperature 1” of the user is equal to or higher than 37.0. When the “body temperature 1” of the user is lower than 37.0, the local controller 7 does not permit passage from the outside to the inside of the management area.

For example, when preventing a disease from spreading outside the management area, a health condition of a user whose body temperature is equal to or higher than 37.5° C. is considered poor. In this configuration, the local controller 7 permits passage from the inside to the outside of the management area when the “body temperature 1” of the user is lower than 37.5. When the “body temperature 1” of the user is equal to or higher than 37.5, the local controller 7 does not permit passage from the outside to the inside of the management area.

For example, when preventing a disease which requires bed rest for a certain period of time from spreading outside the management area, a health condition of a user whose average body temperature was equal to or higher than 37.5° C. on any of the past five days is considered poor. In this configuration, the local controller 7 permits passage from the inside to the outside of the management area when all of the “body temperature 1” to the “body temperature 6” are lower than 37.5. When at least one of the “body temperature 1” to the “body temperature 6” is equal to or higher than 37.5, the local controller 7 does not permit passage from the outside to the inside of the management area.

For example, when preventing a disease from being transmitted outside the management area, a health condition of a user whose body temperature is equal to or higher than 37.0° C. is considered poor. In this configuration, the local controller 7 permits passage from the inside to the outside of the management area when the “body temperature 1” is equal to or higher than 37.0. When the “body temperature 1” is equal to or higher than 37.0, the local controller 7 does not permit passage from the outside to the inside of the management area.

A reference value of body temperature can be changed as appropriate. For example, when influenza that is not accompanied by high fever is going around, the body temperature reference value may be changed from 37.5 to 37.3.

As shown in FIG. 2, the local controller 7 unlocks the electronic lock 1a when permitting passage. As shown in FIG. 3, the local controller 7 issues an alarm instead of unlocking the electronic lock 1a when passage is not permitted. For example, the local controller 7 causes the card reader 5 to emit a warning sound.

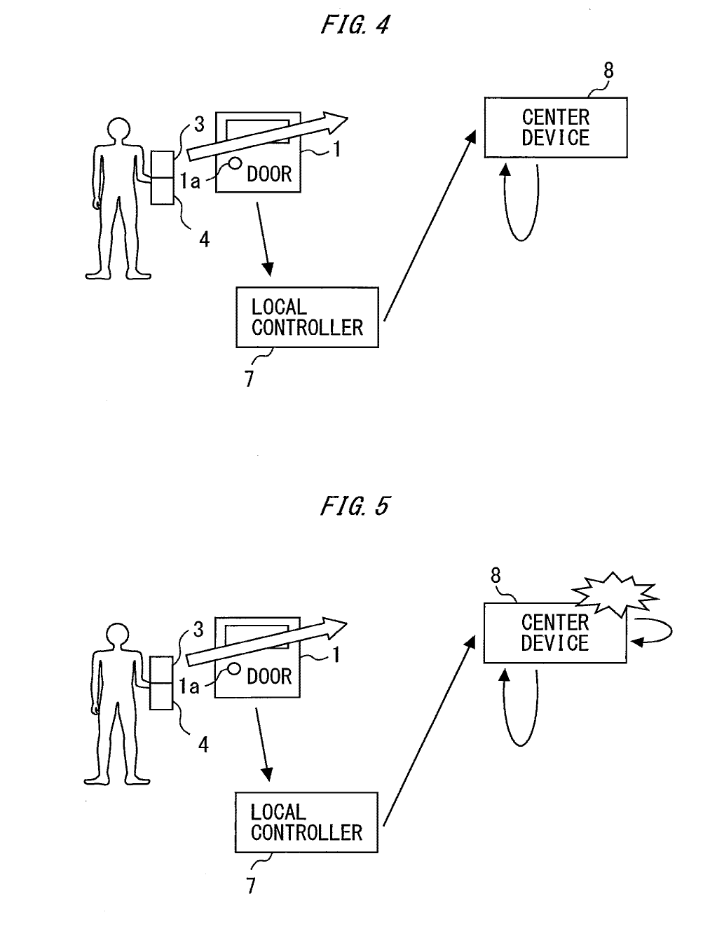

Next, health management of a user will be described with reference to FIGS. 4 and 5.

FIGS. 4 and 5 are diagrams for illustrating health management of a user by the dynamic state management system according to the first embodiment of the present invention.

As shown in FIG. 4, the center device 8 performs health management of a user present inside the management area based on the information stored in the in-room presence information storage device 8c. Specifically, the center device 8 detects a user in a poor health condition inside the management area.

As shown in FIG. 5, when at least one of the number and a percentage of users in a poor health condition inside the management area satisfies a condition set in advance, the center device 8 issues an alarm as an alarm device.

For example, when the number of users in a poor health condition inside the management area reaches a number set in advance, the center device 8 issues an alarm. For example, when the number of users in a poor health condition inside the management area reaches 30, the center device 8 issues an alarm.

For example, when the percentage of users in a poor health condition inside the management area reaches a percentage set in advance, the center device 8 issues an alarm. For example, when the percentage of users in a poor health condition inside the management area reaches 20%, the center device 8 issues an alarm.

For example, when the number of users in a poor health condition inside the management area reaches a number set in advance and the percentage of the users in a poor health condition inside the management area reaches a percentage set in advance, the center device 8 issues an alarm. For example, when the number of users in a poor health condition inside the management area reaches 30 and the percentage of the users in a poor health condition inside the management area reaches 20%, the center device 8 issues an alarm.

For example, when a period of stay by a user in a poor health condition inside the management area reaches a period of stay set in advance, the center device 8 issues an alarm as an alarm device. For example, when the period of stay of a user in a poor health condition inside the management area reaches 30 minutes, the center device 8 issues an alarm.

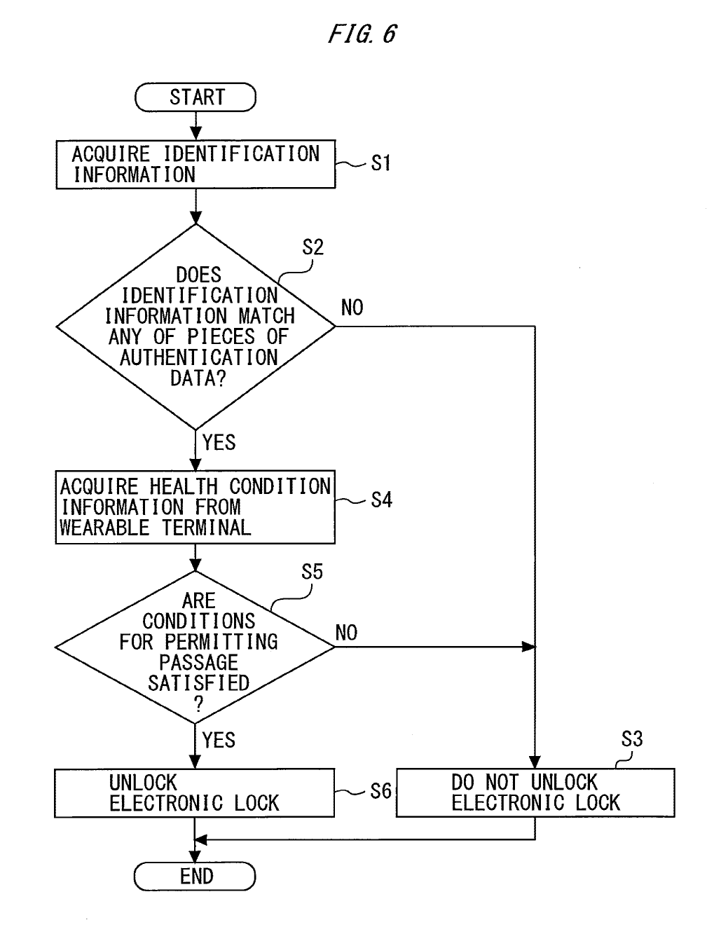

Next, an outline of operations of the local controller 7 will be described with reference to FIG. 6.

FIG. 6 is a flow chart for illustrating an outline of operations of the local controller of the dynamic state management system according to the first embodiment of the present invention.

In step S1, the local controller 7 acquires identification information from the card reader 5. Subsequently, the local controller 7 advances to step S2. In step S2, the local controller 7 determines whether or not the identification information matches any of the pieces of authentication data.

When the identification information does not match any of the pieces of authentication data in step S2, the local controller 7 advances to step S3. In step S3, the local controller 7 does not unlock the electronic lock 1a. Subsequently, the operation ends.

When the identification information matches any of the pieces of authentication data in step S2, the local controller 7 advances to step S4. In step S4, the local controller 7 acquires health condition information from the wearable terminal 4 of a user. Subsequently, the local controller 7 advances to step S5.

In step S5, the local controller 7 determines whether or not conditions for permitting passage through a corresponding entrance are satisfied.

When conditions for permitting passage through a corresponding entrance are not satisfied in step S5, the local controller 7 advances to step S3. In step S3, the local controller 7 does not unlock the electronic lock 1a. Subsequently, the operation ends.

When conditions for permitting passage through a corresponding entrance are satisfied in step S5, the local controller 7 advances to step S6. In step S6, the local controller 7 unlocks the electronic lock 1a. Subsequently, the operation ends.

According to the first embodiment described above, the dynamic state management system makes a determination in accordance with health condition information collected from a wearable terminal of a user. Therefore, the dynamic state management system can operate appropriately according to a health condition of the user.

For example, when health condition information collected from the wearable terminal 4 of a user is information indicating a poor health condition, passage through an entrance from the outside to the inside of a management area is not permitted. In this case, a disease can be prevented from spreading inside the management area.

For example, when health condition information collected from the wearable terminal of a user is information indicating a poor health condition over a certain period in the past, passage through an entrance from the outside to the inside of a management area is not permitted. In this case, a disease which requires bed rest for a certain period of time can be prevented from spreading inside the management area. For example, influenza can be prevented from spreading inside the management area.

For example, when health condition information collected from the wearable terminal 4 of a user is information indicating a good health condition, passage through an entrance from the outside to the inside of a management area is not permitted. In this case, a disease can be prevented from being transmitted inside the management area.

For example, when health condition information collected from the wearable terminal 4 of a user is information indicating a poor health condition, passage through an entrance from the inside to the outside of a management area is not permitted. In this case, a disease can be prevented from spreading outside the management area.

For example, when health condition information collected from the wearable terminal 4 of a user is information indicating a poor health condition over a certain period in the past, passage through an entrance from the inside to the outside of a management area is not permitted. In this case, a disease which requires bed rest for a certain period of time can be prevented from spreading outside the management area. For example, influenza can be prevented from spreading outside the management area.

For example, when health condition information collected from the wearable terminal 4 of a user is information indicating a good health condition, passage through an entrance from the inside to the outside of a management area is not permitted. In this case, a disease can be prevented from being transmitted outside the management area.

In addition, the in-room presence information storage device 8c stores in-room presence info, illation which associates passage information and health condition information with each other. Therefore, health management of users present inside the management area can be performed.

Furthermore, the center device 8 issues an alarm when at least one of the number and a percentage of users in a poor health condition inside the management area satisfies a condition set in advance. Therefore, in accordance with the alarm, users in a good health condition can be prompted to exit the management area. As a result, disease can be prevented from spreading inside the management area.

In addition, the center device 8 issues an alarm when a period of stay by a user in a poor health condition inside the management area reaches a period of stay set in advance. Therefore, in accordance with the alarm, ventilation of the management area can be promoted. As a result, disease can be prevented inside the management area.

Moreover, the gate 2 may be opened when permitting passage. The gate 2 need not be opened when passage is not permitted. Even in this case, the dynamic state management system can operate appropriately according to a health condition of the user.

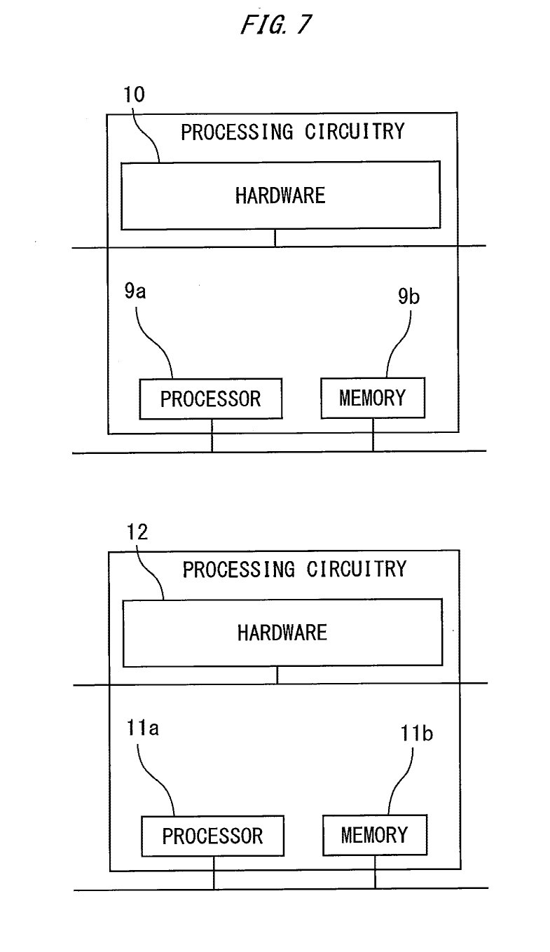

Next, examples of the local controller 7 and the center device 8 will be described with reference to FIG. 7.

FIG. 7 is a hardware configuration diagram of the local controller and the center device of the dynamic state management system according to the first embodiment of the present invention.

Each function of the local controller 7 may be realized by a processing circuitry. For example, the processing circuitry includes at least one processor 9a and at least one memory 9b. For example, the processing circuitry includes at least one piece of dedicated hardware 10.

When the processing circuitry includes at least one processor 9a and at least one memory 9b, each function of the local controller 7 is realized by software, firmware, or a combination of software and firmware. At least one of the software and the firmware is described as a program. At least one of the software and the firmware is stored in the at least one memory 9b. The at least one processor 9a realizes each function of the local controller 7 by reading and executing a program stored in the at least one memory 9b. The at least one processor 9a is also referred to as a CPU (Central Processing Unit), a central processor, a processing unit, an arithmetic unit, a microprocessor, a microcomputer, or a DSP. For example, the at least one memory 9b is a non-volatile or volatile semiconductor memory such as a RAM, a ROM, a flash memory, an EPROM, and an EEPROM, a magnetic disk, a flexible disk, an optical disk, a compact disc, a mini disc, a DVD, or the like.

When the processing circuitry includes at least one piece of dedicated hardware 10, the processing circuitry is, for example, a single circuitry, a composite circuitry, a programmed processor, a parallel-programmed processor, an ASIC, an FPGA, or a combination thereof. For example, each function of the local controller 7 may be realized by a processing circuitry. For example, the respective functions of the local controller 7 may be collectively realized by a processing circuitry.

With respect to the respective functions of the local controller 7, a part thereof may be realized by the dedicated hardware 10 and another part thereof may be realized by software or firmware. For example, a function for acquiring identification information from the card reader 5 may be realized by a processing circuitry as the dedicated hardware 10 and functions other than the function for acquiring identification information from the card reader 5 may be realized by having the at least one processor 9a read and execute a program stored in the at least one memory 9b.

In this manner, the processing circuitry realizes the respective functions of the local controller 7 using the hardware 10, software, firmware, or a combination thereof.

Each function of the center device 8 may be realized by a processing circuitry. For example, the processing circuitry includes at least one processor 11a and at least one memory 11b. For example, the processing circuitry includes at least one piece of dedicated hardware 12.

When the processing circuitry includes at least one processor 11a and at least one memory 11b, each function of the center device 8 is realized by software, firmware, or a combination of software and firmware. At least one of the software and the firmware is described as a program. At least one of the software and the firmware is stored in the at least one memory 11b. The at least one processor 11a realizes each function of the center device 8 by reading and executing a program stored in the at least one memory 11b. The at least one processor 11a is also referred to as a CPU (Central Processing Unit), a central processor, a processing unit, an arithmetic unit, a microprocessor, a microcomputer, or a DSP. For example, the at least one memory 11b is a non-volatile or volatile semiconductor memory such as a RAM, a ROM, a flash memory, an EPROM, and an EEPROM, a magnetic disk, a flexible disk, an optical disk, a compact disc, a mini disc, a DVD, or the like.

When the processing circuitry includes at least one piece of dedicated hardware 12, the processing circuitry is, for example, a single circuitry, a composite circuitry, a programmed processor, a parallel-programmed processor, an ASIC, an FPGA, or a combination thereof. For example, each function of the center device 8 may be realized by a processing circuitry. For example, the respective functions of the center device 8 may be collectively realized by a processing circuitry.

With respect to the respective functions of the center device 8, a part thereof may be realized by the dedicated hardware 12 and another part thereof may be realized by software or firmware. For example, a function for issuing an alarm may be realized by a processing circuitry as the dedicated hardware 12 and functions other than the function for issuing an alarm may be realized by having the at least one processor 11a read and execute a program stored in the at least one memory 11b.

In this manner, the processing circuitry realizes the respective functions of the center device 8 using the hardware 12, software, firmware, or a combination thereof.

### Second Embodiment

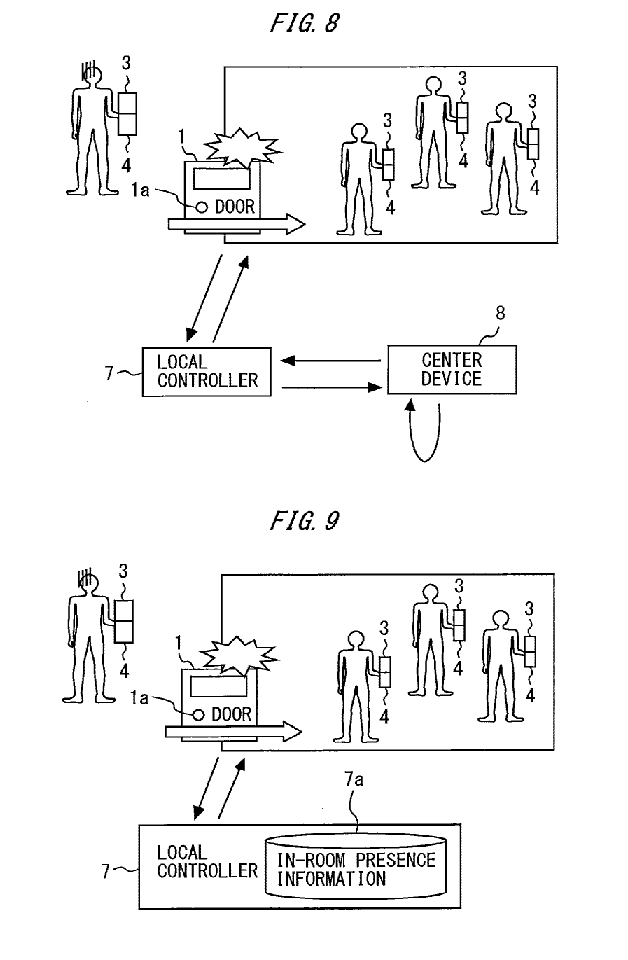

FIG. 8 is a diagram for illustrating a determination whether or not to permit passage by a dynamic state management system according to a second embodiment of the present invention. It should be noted that portions that are the same as or comparable to the first embodiment are assigned same reference signs. Descriptions of such portions will be omitted.

In the second embodiment, the center device 8 transmits health condition information of a user present inside a management area to the local controller 7.

The local controller 7 determines whether or not to permit passage through an entrance from the outside to the inside of the management area based on a health condition collected from the wearable terminal 4 of a user and a health condition of a user present inside the management area.

For example, when health condition information collected from the wearable terminal 4 of a user is information indicating a poor health condition and a user in a good health condition is present inside the management area, the local controller 7 does not permit passage through the entrance from the outside to the inside of a management area.

According to the second embodiment described above, when health condition information collected from the wearable terminal 4 of a user is information indicating a poor health condition and a user in a good health condition is present inside the management area, passage through the entrance from the outside to the inside of a management area is not permitted. In this case, disease can be prevented from spreading inside the management area.

Alternatively, a configuration may be adopted in which, when health condition information collected from the wearable terminal 4 of a user is information indicating a good health condition and a user in a poor health condition is present inside the management area, passage through the entrance from the outside to the inside of the management area is not permitted. In this case, disease can be prevented from spreading inside the management area.

### Third Embodiment

FIG. 9 is a diagram for illustrating a determination whether or not to permit passage by a dynamic state management system according to a third embodiment of the present invention. It should be noted that portions that are the same as or comparable to the first embodiment are assigned same reference signs. Descriptions of such portions will be omitted.

In the third embodiment, the local controller 7 is provided with an in-room presence information storage device 7b that is similar to the in-room presence information storage device 8c.

The local controller 7 assesses a health condition of a user present inside the management area based on the information stored in the in-room presence information storage device 7b. The local controller 7 determines whether or not to permit passage through an entrance from the outside to the inside of the management area based on a health condition collected from the wearable terminal 4 of a user and a health condition of a user present inside the management area.

For example, when health condition information collected from the wearable terminal 4 of a user is information indicating a poor health condition and a user in a good health condition is present inside the management area, the local controller 7 does not permit passage through the entrance from the outside to the inside of a management area.

According to the third embodiment described above, when health condition information collected from the wearable terminal 4 of a user is information indicating a poor health condition and a user in a good health condition is present inside the management area, passage through the entrance from the outside to the inside of a management area is not permitted. In this case, disease can be prevented from spreading inside the management area.

Alternatively, a configuration may be adopted in which, when health condition information collected from the wearable terminal 4 of a user is information indicating a good health condition and a user in a poor health condition is present inside the management area, passage through the entrance from the outside to the inside of the management area is not permitted. In this case, disease can be prevented from spreading inside the management area.

### Fourth Embodiment

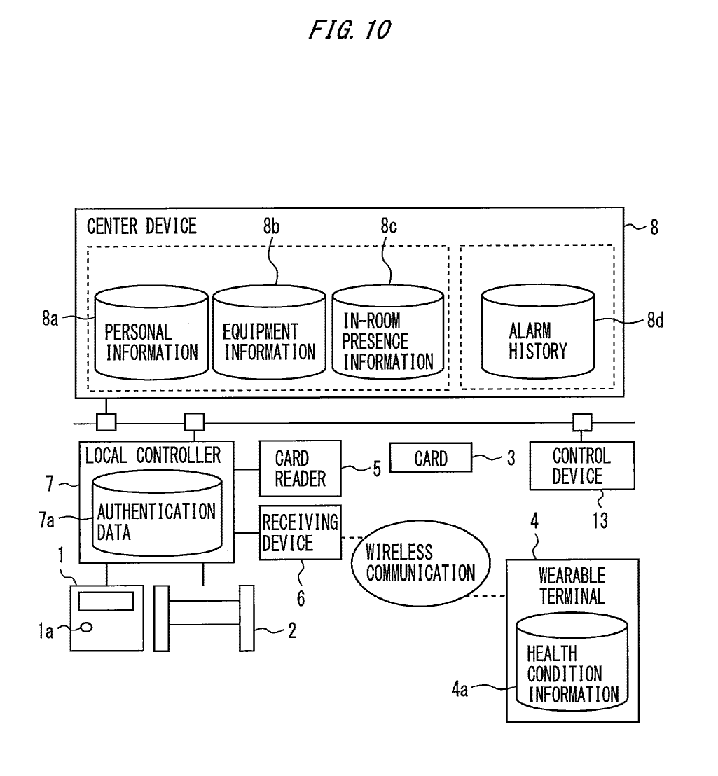

FIG. 10 is a diagram for illustrating an elevator system according to a fourth embodiment of the present invention. FIG. 11 is a diagram for illustrating an outline of control by the elevator system according to the fourth embodiment of the present invention. It should be noted that portions that are the same as or comparable to the first embodiment are assigned same reference signs. Descriptions of such portions will be omitted.

In the fourth embodiment, each of a plurality of card readers 5 is provided on each of a plurality of halls of an elevator. Note that only one card reader 5 is shown in FIGS. 10 and 11.

Each of a plurality of receiving devices 6 is provided on each of the plurality of halls of the elevator. Note that only one receiving device 6 is shown in FIGS. 10 and 11.

For example, a control device 13 is provided in a machine room (not shown) of the elevator. For example, the control device 13 is provided in a hoistway (not shown) of the elevator.

When the user waves the card 3 over the card reader 5 on a hall, the card reader 5 reads identification information from the card 3. The local controller 7 collates the identification information read by the card reader 5 with authentication data.

When the user approaches the entrance of a hall, the receiving device 6 receives health condition information from the wearable terminal 4 of the user.

When the identification information read by the card reader 5 matches any of the pieces of authentication data, the local controller 7 acquires health condition information of a user from the receiving device 6. The local controller 7 transmits information on a car arrangement request on the hall and health condition information to the control device 13.

For example, the control device 13 registers a call from the hall in accordance with information on a car arrangement request on the hall. For example, the control device 13 determines a car to respond to the call from the hall in accordance with health condition information of a user on the hall. For example, the control device 13 acquires health condition information of a user inside a car based on the information stored in the in-room presence information storage device 8c. For example, the control device 13 determines a car to respond to the call from the hall in accordance with health condition information of a user inside a car. In doing so, the control device 13 determines a health condition of a user based on conditions set in advance.

For example, when a first condition is set, the control device 13 determines a health condition of a user based on whether or not a condition where the “body temperature 1” of the user is equal to or higher than 37.5 is satisfied. Specifically, the control device 13 determines that a health condition of a user whose “body temperature 1” is equal to or higher than 37.5 is poor. The control device 13 determines that a health condition of a user whose “body temperature 1” is lower than 37.5 is good.

For example, when a second condition is set, the control device 13 determines a health condition of a user based on whether or not a condition where all of the “body temperature 1” to the “body temperature 6” of the user is lower than 37.0 is satisfied. Specifically, the control device 13 determines that a health condition of a user of which at least one of the “body temperature 1” to the “body temperature 6” is equal to or higher than 37.0 is poor. The control device 13 determines that a health condition of a user of which all of the “body temperature 1” to the “body temperature 6” is lower than 37.0 is good.

A reference value of body temperature can be changed as appropriate. For example, when influenza that is not accompanied by high fever is going around, the body temperature reference value may be changed from 37.5 to 37.3.

For example, when the first condition is set, the control device 13 causes a car to preferentially respond to a call from a hall corresponding to a user in a poor health condition.

For example, when the second condition is set, the control device 13 does not cause a car carrying a user in a poor health condition to respond to a call from a hall corresponding to a user in a good health condition. The control device 13 does not cause a car carrying a user in a good health condition to respond to a call from a hall corresponding to a user in a poor health condition.

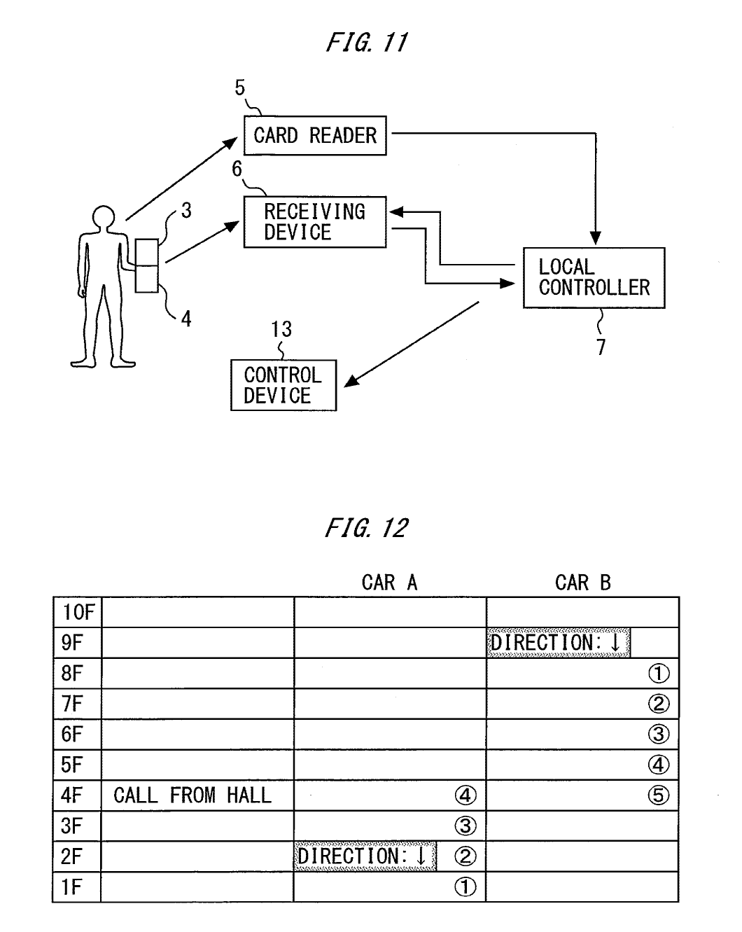

Next, operations of a car when the first condition is set will be described with reference to FIG. 12.

FIG. 12 is a diagram for illustrating operations of a car of the elevator system according to the fourth embodiment of the present invention.

In FIG. 12, a car A is stopped on a second floor. A direction of travel of the car A is set downward. A car B is stopped on a ninth floor. A direction of travel of the car B is set downward.

In this state, when a call from a hall corresponding to a user in a poor health condition is registered at the fourth floor, the car A with a shortest waiting time with respect to the call from the hall responds to the hall call.

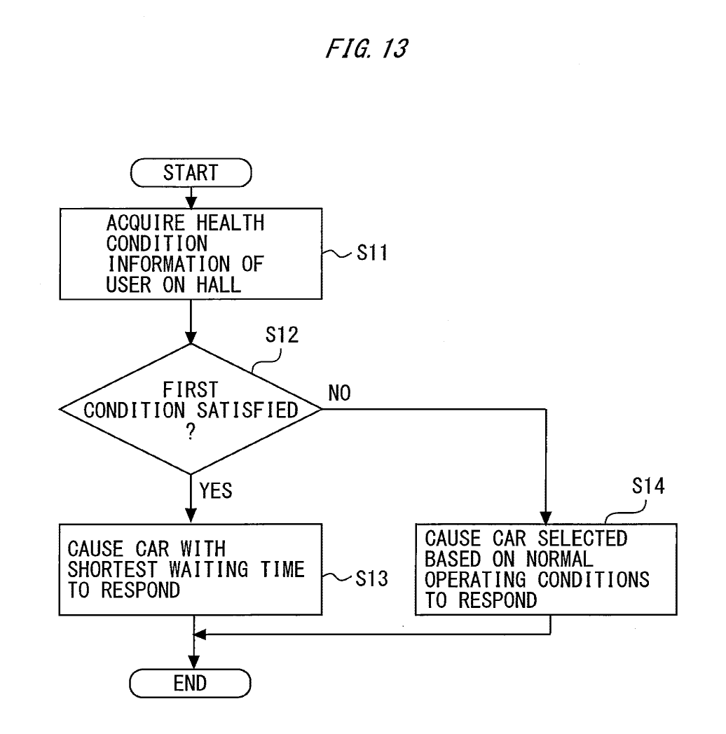

Next, operations of the control device 13 when the first condition is set will be described with reference to FIG. 13.

FIG. 13 is a diagram for illustrating operations of the control device of the elevator system according to the fourth embodiment of the present invention.

In step S11, the control device 13 acquires health condition information of a user on a hall. Subsequently, the control device 13 advances to step S12. In step S12, the control device 13 determines whether or not the first condition is satisfied.

When the first condition is satisfied in step S12, the control device 13 advances to step S13. In step S13, the control device 13 causes a car with a shortest waiting time to respond to a call from the hall. Subsequently, the operation ends.

When the first condition is not satisfied in step S12, the control device 13 advances to step S14. In step S14, the control device 13 causes a car selected based on normal operating conditions to respond to a call from the hall. Subsequently, the operation ends.

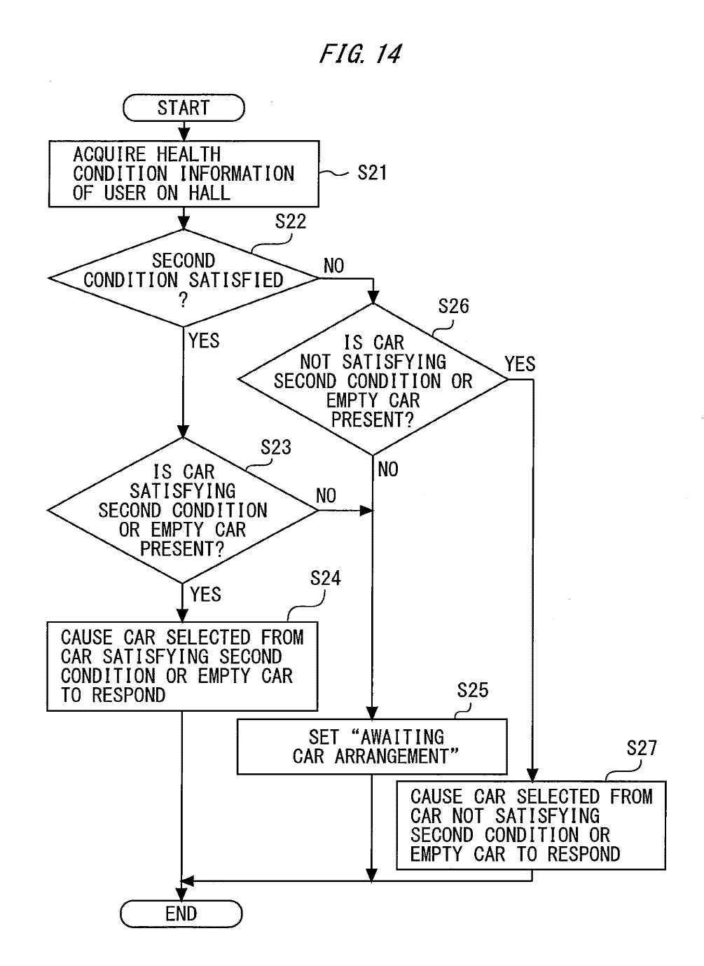

Next, operations of the control device 13 when the second condition is set will be described with reference to FIG. 14.

FIG. 14 is a diagram for illustrating operations of the control device of the elevator system according to the fourth embodiment of the present invention.

In step S21, the control device 13 acquires health condition information of a user on a hall. Subsequently, the control device 13 advances to step S22. In step S22, the control device 13 determines whether or not the second condition is satisfied.

When the second condition is satisfied in step S22, the control device 13 advances to step S23. In step S23, the control device 13 determines whether or not either a car in which the second condition is satisfied in all health conditions of a user inside the car or an empty car is present.

When either a car in which the second condition is satisfied in all health conditions of a user inside the car or an empty car is present in step S23, the control device 13 advances to step S24. In step S24, the control device 13 causes a car selected from a car in which the second condition is satisfied in all health conditions of a user inside the car or an empty car to respond to the call from the hall. Subsequently, the operation ends.

When neither a car in which the second condition is satisfied in all health conditions of a user inside the car nor an empty car is present in step S23, the control device 13 advances to step S25. In step S25, the control device 13 sets “awaiting car arrangement”. In this case, the control device 13 waits until either a car in which the second condition is satisfied in all health conditions of a user inside the car or an empty car becomes available. Subsequently, the operation ends.

When the second condition is not satisfied in step S22, the control device 13 advances to step S26. In step S26, the control device 13 determines whether or not either a car in which the second condition is not satisfied in all health conditions of a user inside the car or an empty car is present.

When either a car in which the second condition is not satisfied in all health conditions of a user inside the car or an empty car is present in step S26, the control device 13 advances to step S27. In step S27, the control device 13 causes a car selected from a car in which the second condition is not satisfied in all health conditions of a user inside the car or an empty car to respond to the call from the hall. Subsequently, the operation ends.

When neither a car in which the second condition is not satisfied in all health conditions of a user inside the car nor an empty car is present in step S26, the control device 13 advances to step S25. In step S25, the control device 13 sets “awaiting car arrangement”. In this case, the control device 13 waits until either a car in which the second condition is not satisfied in all health conditions of a user inside the car or an empty car becomes available. Subsequently, the operation ends.

According to the fourth embodiment described above, the control device 13 controls an elevator based on information on a car arrangement request on a relevant hall and on health condition information. Therefore, the elevator system can operate appropriately according to a health condition of a user.

In addition, the control device 13 causes a car to preferentially respond to a call from a hall corresponding to a user in a poor health condition. Therefore, a sick user can be preferentially transported in an elevator.

Furthermore, when received health condition information is information indicating a poor health condition and a user in a good health condition is present inside a car, the control device 13 does not cause the car to respond to a call from a hall on which the card reader 5 having read identification information from the card 3 of a user in a poor health condition is present. Therefore, disease can be prevented from being transmitted inside the car.

In addition, when received health condition information is information indicating a good health condition and a user in a poor health condition is present inside a car, the control device 13 does not cause the car to respond to a call from a hall on which the card reader 5 having read identification information from the card 3 of a user in a good health condition is present. Therefore, disease can be prevented from being transmitted inside the car.

Moreover, in the first to fourth embodiments, health condition information of the wearable terminal 4 can be transmitted to the local controller 7 via a public line. In this case, the local controller 7 determines whether or not to permit passage through an entrance based on health condition information stored in the local controller 7 itself. Therefore, whether or not to permit passage through an entrance can be determined without having to use the receiving device 6.

In addition, in the first to fourth embodiments, health condition information of the wearable terminal 4 can be transmitted to a cloud server (not shown) via a public line. In this case, the local controller 7 determines whether or not to permit passage through an entrance based on health condition information stored in the cloud server. Therefore, whether or not to permit passage through an entrance can be determined without having to use the receiving device 6.

### INDUSTRIAL APPLICABILITY

As described above, the dynamic state management system and the elevator system can be utilized in a system which operates appropriately according to a health condition of a user.

### REFERENCE SIGNS LIST

1 Door
1a Electronic lock
2 Gate
3 Card
4 Wearable terminal
4a Storage unit
5 Card reader
6 Receiving device
7 Local controller
7a Authentication data storage device
7b In-room presence information storage device
8 Center device
8a Personal information storage device
8b Equipment information storage device
8c In-room presence information storage device
8d Alarm history storage device
9a Processor
9b Memory
10 Hardware
11a Processor
11b Memory
12 Hardware
13 Control device

1-12. (canceled)

13. An elevator system, comprising:
a plurality of reading devices provided on respective halls of an elevator, the plurality of reading devices reading identification information from an identifier of a user; and
a control device configured to control the elevator based on the identification information read by any reading device of the plurality of reading devices and health condition information collected from a wearable terminal of a user corresponding to the identification information,
wherein the control device, when health condition information collected from a wearable terminal of a user corresponding to the identification information read by any reading device of the plurality of reading devices is information indicating a poor health condition, causes a car with a shortest waiting time to respond to a call from a hall on which the reading device is present.

14-16. (canceled)
